(19) United States
(12) Patent Application Publication
(10) Pub. No.: US 2025/0234907 A1
(43) Pub. Date: Jul. 24, 2025

(54) METHOD FOR PRODUCING CELL-BASED FISH CONTAINING FISH MUSCLE SHAPE-MIMICKING MUSCLE USING 3D BIOPRINTING TECHNOLOGY AND CELL-BASED FISH PREPARED THEREBY

(71) Applicant: Baobab healthcare Inc., Ansan-si (KR)

(72) Inventors: Jeong-Seok LEE, Ansan-si (KR); Gi Hoon YANG, Ansan-si (KR); Donggu KANG, Ansan-si (KR); Eunjoeng CHOI, Ansan-si (KR); Yoonju NAM, Ansan-si (KR); Kyung-Hyun Min, Ansan-si (KR); Hojun JEON, Ansan-si (KR)

(21) Appl. No.: 18/290,336

(22) PCT Filed: Dec. 5, 2022
(86) PCT No.: PCT/KR2022/019580

§ 371 Date: Nov. 13, 2023

(30) Foreign Application Priority Data
Oct. 13, 2022 (KR) 10-2022-0131399

(51) Int. Cl. A23L 17/00; A23P 20/20; A23P 20/25; B33Y 10/00; B33Y 30/00; B33Y 80/00; C12N 5/071; C12N 5/077

(52) U.S. Cl. CPC A23L 17/00; A23P 20/20; B33Y 10/00; B33Y 30/00; B33Y 80/00; C12N 5/0653; C12N 5/0658; C12N 5/069; A23P 2020/253

(57) ABSTRACT

Provided are a method for producing an edible cell-based fish that can realize actual fish muscle shape and texture, and a cell-based fish prepared thereby. The method for producing cell-based fish containing fish muscle shape-mimicking muscle using 3D bioprinting technology includes the steps of: mixing cells for cultured fish and an edible bioink; printing the cell printing ink in a fish muscle-mimic pattern to form a laminated structure; adding a crosslinking agent to the laminated structure; and culturing the laminated structure to which the crosslinking agent is added into a fish muscle tissue.

### TECHNICAL FIELD

The present disclosure relates to a method for producing cell-based fish containing a fish muscle shape-mimicking muscle using 3D bioprinting technology, and a cell-based fish produced thereby.

### BACKGROUND ART

By the year 2050, it is expected that the world population will reach nearly 9.8 billion people, and people will consume 450 million tons of animal protein per year (FAO, 2019), thereby facing a protein crisis. Also, because of a decrease in agricultural land and crop yields caused by climate change such as global warming, a mass death of livestock caused by coronavirus infection, a rapid increase in grain prices caused by war, and the like, the production costs increase relative to the demand for cereals, and the production area decreases, whereby it is expected that livestock products become increasingly expensive foods.

On the other hand, marine seafood, represented by fish, are the optimal protein source that can replace poultry, beef, and pork. It has a higher essential protein content and lower fat content than meat, and contains more vitamins D, E, and minerals than meat. However, because of adverse effects on health caused by microplastics, problems of exposure to radioactive contaminated water caused by nuclear power plant accidents, etc., problems of heavy metal accumulation, extinction of fish caused by changes in seawater temperature, the reduction in fish populations caused by bycatch and overfishing, it is expected that marine seafood is increasingly polluted and diminished.

Cell-based fishes means edible fishes obtained by mass-culturing living fish cells in a research environment, rather than fisheries or aquaculture. This is marine seafood made by culturing cells, and is called cell-based seafood or cell-based seafood. At this time, fish cells mean muscle stem cells or myoblast cells that constitute flesh meat.

However, the cell-based fish production techniques known to date mostly utilize scaffolds that carry cells. Scaffold production technology consists of non-uniform pores by salt leaching and gas forming, and cells are seeded thereon, which is finally organized into uneven muscle tissue. Additionally, it is not possible to mimic the complex microstructure of muscle.

Therefore, in order to solve future food security and eliminate consumer rejection, there is a need to develop a technology that can mimic actual fish muscle shape and texture.

### DETAILED DESCRIPTION OF THE INVENTION

### Technical Problem

One embodiment aims at providing a method for producing an edible cell-based fish that can realize actual fish muscle shape and texture.

Another embodiment aims at providing a cell-based fish having actual fish muscle shape and texture.

### Technical Solution

According to one embodiment, there is provided a method for producing cell-based fish containing fish muscle shape-mimicking muscle using 3D bioprinting technology, the method comprising the steps of: mixing cells for cultured fish and an edible bioink to prepare a cell printing ink; printing the cell printing ink in a fish muscle-mimic pattern to form a laminated structure; adding a crosslinking agent to the laminated structure; and culturing the laminated structure to which the crosslinking agent is added into a fish muscle tissue.

According to another embodiment, there is provided a cell-based fish with layer-by-layer assembled muscle tissue containing fish muscle-derived cells.

### Advantageous Effects

The method for producing cell-based fish according to one embodiment can provide an edible cell-based fish that can align muscle cells so as to mimic the ‘W’ shape of a real fish muscle, thereby realizing actual fish muscle shape and texture.

Conventionally, when a porous support (scaffold) is used, there is a problem that the porosity is large, the mechanical properties are deteriorated, and the space where cells can attach depends on the pores in the support, and also there is a problem that the muscle content in the cell-based fish that is ultimately produced is limited. According to one embodiment, however, cells and edible bioink are mixed to prepare a cell printing ink, and then this ink is used to print and then culture, so that the cell content can be adjusted, and the cells are surrounded by an extracellular matrix to provide an environment similar to that of actual fish. Therefore, it is possible to mimic the texture similar to actual fish.

Further, the method for producing cell-based fish according to one embodiment can easily adjust the cell environment by adjusting the content of the edible bioink.

In addition, during printing ejection for producing cell-based fish according to one embodiment, the inner diameter of the printer nozzle, printing pneumatic pressure, and/or viscosity of the bioink are controlled so that a desired shear stress can be applied to a specimen, thereby accelerating cell alignment to produce a cell-based fish that mimics actual fish muscle shape and texture.

### BRIEF DESCRIPTION OF THE DRAWINGS

FIG. 1 is an exemplary diagram showing a basic combination of printing patterns for mimicking fish muscle tissue.

FIGS. 2a to 2c are exemplary diagrams showing examples of laminating patterns in the LBL printing schemes for mimicking fish muscle tissue.

FIG. 3 is an exemplary diagram of an off-set lamination method suitable for mimicking the round shape of fish.

FIG. 4 shows muscle stem cells extracted from Sebastes schlegeli (Korean rockfish).

FIG. 5 is images showing the morphological appearance of the cell-based fish laminated structure output by bioprinting.

FIG. 6 is an image of cell-based fish in which muscle tissue is generated after cell culture for 4 weeks.

FIG. 7 shows images of cell-based fishes after cooking.

FIG. 8 is a fluorescence image showing the difference between scaffold culture and cell printing culture of muscle stem cells.

FIG. 9 shows the results of measuring the aspect ratio of cells formed according to scaffold culture and cell printing culture of muscle stem cells.

FIG. 10 shows the results of measuring the full width at half maximum of cells formed according to scaffold culture and cell printing culture of muscle stem cells.

FIG. 11 shows the results of measuring the mRNA expression levels of Myogenin, MyoD, and MyHC in cell-based fishes formed according to scaffold culture and cell printing culture.

FIG. 12 shows fluorescence images measured on the 1st day and the 4th day of culture after varying the composition of the edible bioink.

FIG. 13 is a graph showing the measured values of compressive stress and tensile strain depending on the printing type of the edible bioink.

FIG. 14 shows fluorescence images measured on the 3rd day and 7th day of culture after forming a laminated structure by varying the cell concentrations.

FIG. 15 shows fluorescence images measured on the 3rd day and 7th day of culture after forming a laminated structure by varying the pneumatic pressure during bioprinting.

### DETAILED DESCRIPTION OF THE EMBODIMENTS

Hereinafter, exemplary embodiments of the present disclosure will be described in more detail with reference to the accompanying drawings so as to be easily realized by those skilled in the art. The present disclosure may, however, be embodied in different forms and should not be constructed as limited to the embodiments set forth herein.

Now, a method for producing cell-based fish according to one embodiment will be described.

For the production of cell-based fish, first, cells for cultured fish and an edible bioink are mixed to prepare a cell printing ink.

The cells for cultured fish may include muscle stem cells (myosatellite cells) or myoblast cells of fishes to be cultured. Muscle stem cells or myoblast cells can be purchased from the outside and used. If necessary, muscle stem cells or myoblast cells can be extracted and used from the white flesh of fishes to be cultured. As an example, the white flesh of fishes to be cultured is isolated, carried on a culture medium, washed, centrifuged to remove an upper layer liquid, and a lower layer liquid is decomposed with collagenase. The decomposition product may be centrifuged to remove the upper layer liquid, and the lower layer liquid may be carried on a culture medium and cultured to extract muscle stem cells or myoblast cells for use.

In order to make the texture of cell-based fish more similar to that of actual fish, the cells for cultured fish may further include fish adipose-derived cells or fish blood vessel-derived cells. For example, it may further include one or more cells selected from the group consisting of adipose stem cells, adipose cells, vascular endothelial stem cells, and vascular endothelial cells. The adipose stem cells, adipose cells, vascular endothelial stem cells, and vascular endothelial cells can also be purchased from the outside and used like muscle stem cells or myoblast cells, or extracted directly from fish adipose or blood vessels and used.

The edible bioink may include alginate.

Alginate is a polysaccharide, which is extracted from algae such as sea mustard and kelp, and has excellent biocompatibility and low toxicity, and is inexpensive. In particular, alginate is easily and quickly hydro-gelled through ionic crosslinking with calcium (Ca²⁺), barium (Ba²⁺), strontium (Sr²⁺), magnesium (Mg²⁺), manganese (Mn²⁺), and zinc (Zn²⁺), which are divalent cations. Therefore, alginate has the function of immediately maintaining the structure of cell-based fish through ionic crosslinking just after printing.

The edible bioink may, in addition to alginate, further include one or more substances selected from the group consisting of collagen, gelatin, and chitosan.

Alginate, which is a polysaccharide, does not have cell adhesion molecule and is negatively charged, so that cells can grow through aggregation without attaching to alginate. Therefore, it is preferable to mix substances that can increase cell adhesion.

Gelatin has the property of changing into a liquid at about 37° C. or more and changing into a hydrogel at about 25° C. or less. In addition, gelatin contains all essential amino acids except tryptophan and cystine, and thus has high nutritional value. Therefore, when mixed with alginate, it can improve cell proliferation, adhesion, and mobility, and may aid in organizing cells into tissues.

Collagen or chitosan can also impart the same efficacy as gelatin. In other words, when mixed with alginate, it can stably maintain the structure by ionic crosslinking of alginate, and can improve cell proliferation, adhesion, and mobility and thus can aid in organizing cells into tissues.

As collagen, atelocollagen or collagen peptide may be used.

In one embodiment, alginate may be used in an amount of 1˜5 w/v %.

The concentration of gelatin may be 1 to 30 w/v % and/or the concentration of collagen may be 1 to 10 w/v %.

Additionally, the edible bioink may further include a pH adjuster. The pH of the edible bioink may be 7.0 to 7.5.

When preparing a cell printing ink by mixing cells for cultured fish and edible bioink, the cells can be mixed in the edible bioink at a concentration of 1×10⁶ cells/mL to 1×10¹⁰ cells/mL. If the concentration is lower than 1×106 cells/mL, the spacing between cells is wide, which makes it difficult to achieve sufficient organization. If the concentration is higher than 1×10¹⁰ cells/mL, the volume of the cells is larger than the volume of the hydrogel that must be mixed, which makes the mixing difficult at corresponding concentrations.

The viscosity of the cell printing ink can increase proportionally to the final concentration of the edible bioink and the number of cells. Therefore, by controlling the final concentration and cell number of the edible bioink, the viscosity of the cell printing ink can be controlled.

Then, the cell printing ink is printed layer by layer (LBL) to form a laminated structure.

The laminated structure may be more suitable for mimicking fish muscle tissue, which is formed from a structure in which pattern A and pattern B, which are different from each other, are laminated, instead of being laminated with only a single pattern. In the lamination scheme, pattern A and pattern B may be alternately laminated, or repeatedly laminating one pattern two or more times and then repeatedly laminating the other pattern two or more times may be alternately performed. In this case, adjacent patterns A may be more suitable for mimicking fish muscle when they are formed offset from each other. Therefore, the meaning of layer-by-layer can be interpreted to include the case where different pattern layers exist between the same pattern layers, in addition to the case where the layer consists of only the same pattern layer.

As an example, pattern A and pattern B may form a grid structure.

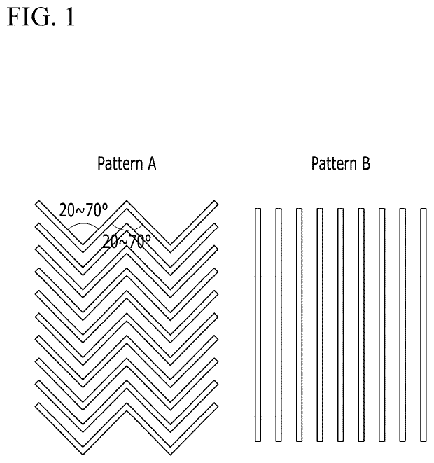

As another example, as illustrated in FIG. 1, pattern A may be formed of a fish muscle tissue mimic pattern in a W shape, and pattern B may be formed of a line pattern that intersects with pattern A. During culture after printing, pattern A (W pattern) is differentiated into a muscle tissue layer, and pattern B is differentiated into a connective tissue layer, thereby capable of producing the shape and texture of an actual fish. The angle of pattern A of the cell-based fish may be 20 to 70 degrees, and the spacing between patterns may be 0.5 mm to 3 mm.

Unlike livestock, the muscle tissue of fish is softer than meat because the muscle fibers are shorter and thicker and the connective tissue between muscle and bone is weak. At this time, when the fish is viewed from the side, a myotome, which is one individual muscle, is folded into a ‘W’ shape, or zigzag shape. A myotome, which is an individual muscle immediately after, are connected by connective tissue (myosepta), and finally the myotome is connected continuously in a zigzag pattern. At this time, when the fish is viewed from the front, the myotomes are not aligned with each other but overlap diagonally in a pyramid structure. This is because the fish itself has an oval structure. Therefore, the shape and size of the myotomes are largest in the torso and become smaller toward the head and tail. This geometric structure creates undulatory motions that allow the fish to progress efficiently. Therefore, in order for the cell-based fish to produce the texture of real fish, it must be able to mimic the geometry of the “W” shape and the continuous slope of each muscularity. Therefore, as illustrated in FIG. 1, pattern A is formed of a W-shaped pattern that mimics fish muscle tissue, and pattern B is formed of a line pattern that intersects with pattern A, thereby allowing it to be a mimic shape of connective tissue.

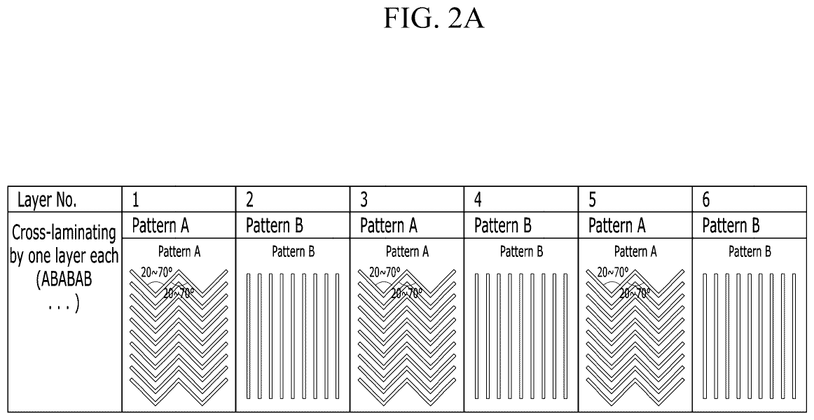

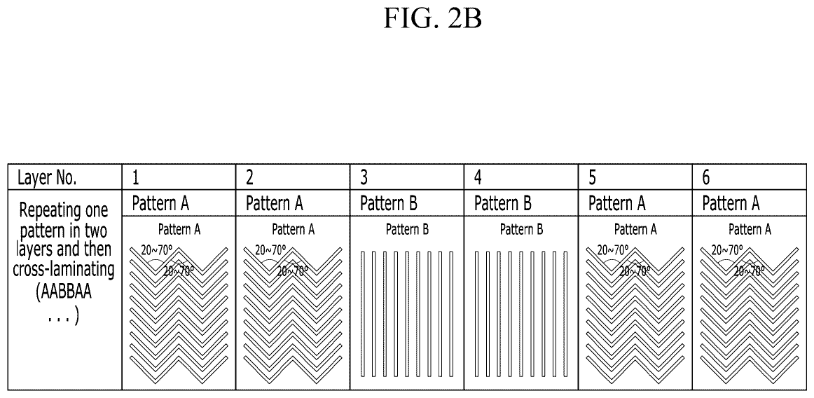

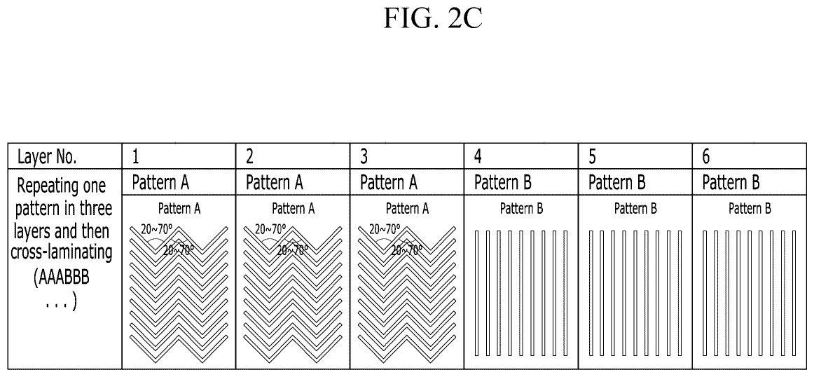

FIG. 2 shows various LBL printing schemes for pattern A and pattern B. Although six layers are illustrated in FIG. 2, but the number of layers is not limited, and can be adjusted depending on the thickness of the cell-based fish to be formed. For example, it can be produced by laminating up to 50 layers. It can be modified in various ways such as cross-laminating by one layer each (ABABAB) (FIG. 2a), repeating one pattern in two layers and then cross-laminating (AABBAA) (FIG. 2b), or repeating one pattern in three layers and then cross-laminating (AAABBB) (FIG. 2c).

It is preferable to start lamination from pattern A (W pattern). This is because when acquiring the structure after final lamination, the cross-sectional area of pattern A is higher than pattern B and can be acquired stably.

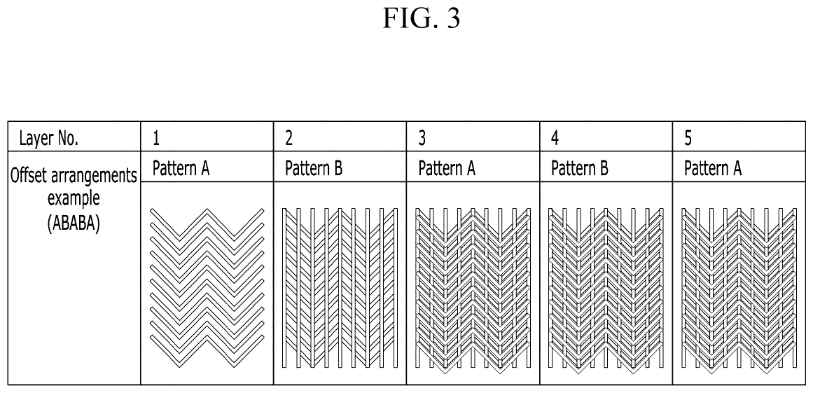

On the other hand, as illustrated in FIG. 3, to mimic the oval shape of a fish, adjacent identical patterns may be arranged offset. For example, pattern A (1) of the lower layer and pattern A (2) of the next layer can be laminated with their starting points being crossed each other by 0.5 mm to 1 mm. Thereby, it is possible to produce a cell-based fish with a texture similar to that of fish.

In one embodiment, the inner diameter of the nozzle of the printer used for 3D printing may be 18G to 32G or 1.00 mm to 0.10 mm. At this time, the printable pneumatic pressure may be 2 kPa to 500 kPa. The X and Y axis feed speed of the printer may be 50 mm/min to 500 mm/min. The temperature range of the printer head may be −10° C. to +40° C. The temperature range of the printing base may be −10° C. to +40° C. These parameters may be parameters that can be variously adjusted depending on the viscosity of the cell printing ink and the thickness and texture of the cell-based fish to be produced. Among these, the printer's nozzle inner diameter and printing pneumatic pressure can be controlled along with the viscosity of the cell printing ink to apply the desired shear stress to the specimen, thereby producing a laminated structure that accelerates cell alignment to form a cell-based fish that mimics fish muscle shape and texture.

In one embodiment, the shape of the cell-based fish can be freely printed, and can be produced in free shapes such as square, rectangular, circular, fish model for infant food, dinosaur model, etc.

Then, a crosslinking agent is added to the laminated structure.

A crosslinking agents can be added to maintain the structure of cell-based fish. The crosslinking agents may be a crosslinking agent containing one or more selected from the group consisting of calcium chloride (CaCl₂)), magnesium chloride (MgCl₂), calcium phosphate (CaP), calcium carbonate (CaCO₂), barium chloride (BaCl₂) and strontium chloride (SrCl₂).

Finally, the laminated structure prepared by adding a crosslinking agent is cultured into a fish muscle tissue.

The culturing step includes a step of aligning the cells in the direction in which they are ejected within the edible bioink and a step of differentiating the cells to form a muscle tissue.

The culture medium used during culture can be a general growth medium for stem cells, and can be suitably modified for culturing the cell-based fish.

As a result, a cell-based fish containing W-shaped muscle tissue that mimics the fish muscle shape is completed.

The cell aspect ratio of muscle stem cells aligned within the cell-based fish can be calculated as the maximum length/minimum length of the cell nucleus or the maximum length/minimum length of the cytoplasm. For example, the cell aspect ratio of the nuclei of aligned muscle stem cells may be 2.0 to 5.0. The cell aspect ratio of the cytoskeleton or cytoplasm of the aligned muscle stem cells may be 3.0 to 10.0.

The aligned directionality of muscle stem cells can be calculated by the full width at half maximum (FWHM), and the full width at half maximum of aligned muscle stem cells may be 1° to 20°. The full width at half maximum refers to the spectrum width at the ½ position of the peak value on the cell alignment angle spectrum.

The degree of differentiation of muscle stem cells can be confirmed by the presence or absence of expression of the Myogenin gene, MyoD (myoblast determination protein gene), or MyHC (Myosin heavy chain) gene.

Hereinafter, the embodiments of the invention described above will be explained in more detail with reference to examples. However, the following examples are for illustrative purposes only and are not intended to limit the scope of the present disclosure. The method for producing cell-based fish according to the present disclosure will be described in detail.

### Production of Cell-Based Fish Containing Fish Muscle Shape-Mimicking Muscles Using 3D Bioprinting Technology

### Cell Culture and Cell Preparation Steps

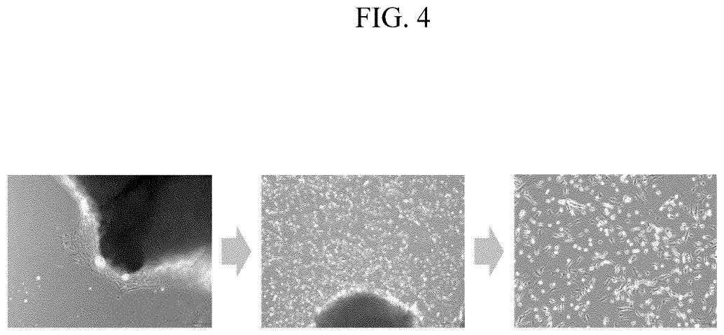

The white meat was isolated from the back of Sebastes schlegeli, and carried on a mixed culture medium, in which DMEM/Nutrient Mixture F-12 Ham (DMEM/F12, Gibco™, 11320033) culture medium and Leibovitz's L-15 Medium (L-15, Gibco™, 11415064) culture medium were mixed in a ratio of 1:1 and which contains 10 mM HEPES (Gibco™, 15630080) and 5% PenStrep (Gibco™, 15140122), at 4° C. for 10 minutes. Next, it was washed with 70% ethanol at 4° C. for 30 seconds, and washed three times with PBS (Phosphate-Buffered Saline) containing 5% PenStrep. It was cut into a size of 0.5˜3 mm³ using a medical knife, and then carried on a mixed culture medium further containing Amphotericin B (Sigma Aldrich, PHR1662) at a concentration of 0.25 μg/mL and Gentamicin (Sigma Aldrich, G1397) at a concentration of 75 μg/mL, and centrifuged at a temperature of 4° C. and a speed of 100 g. After centrifugation, the upper layer liquid was removed, and this was repeated four times. The tissue was then carried on a mixed culture medium containing Collagenase I (Gibco™, 17018029) at a concentration of 625 IU/m at room temperature for 1 hour. After that, centrifugation was performed at a temperature of 4° C. and a speed of 100 g to remove an upper layer liquid, and this was repeated four times. The tissue was carried on a mixed culture medium in which DMEM/F12 medium and L-15 medium were mixed in a ratio of 1:1 and which further contains 1% MEM Non-Essential Amino Acids Solution (Gibco™, 11140050), 20% FBS (Gibco™, 16000044), and bFGF (1 ng/mL, Gibco™, PHG0311). Then, the tissue was attached to a T-flask, the mixed medium was removed, and it was allowed to dry for 30 minutes. Then, the mixed medium was filled and cultured at a temperature of 24° C. for 18 hours, and then 100% of the culture medium was added and cultured for 3 days. Then, 50% of the mixed medium was removed at two-day intervals, 50% of the new mixed medium was added, and the cells were cultured for 7 days to extract muscle stem cells. The muscle stem cells extracted from Sebastes schlegeli is illustrated in FIG. 4.

### Step of Preparing Bioink and Cell Printing Ink

An edible bioink was prepared using alginate, gelatin, and collagen peptide. 3% alginate, 5% gelatin, 10% collagen peptide, and 3% binder were completely dissolved using a portable water on a stirrer for over 24 hours to prepare a bioink, and titrated to pH 7.4 using NaHCO₃. Then, the cells were physically mixed with the bioink at a concentration of 1×10⁸ cells/mL to prepare a cell printing ink.

### Step of Printing Laminated Structure Using a 3D Printer

Cell-based fish structures were fabricated using the Solid Works 3D rendering program. Cell-based fishes were produced using a 3D printer, wherein the structures were fabricated by appropriately adjusting the pressure, printing speed, temperature, ink concentration, and the like.

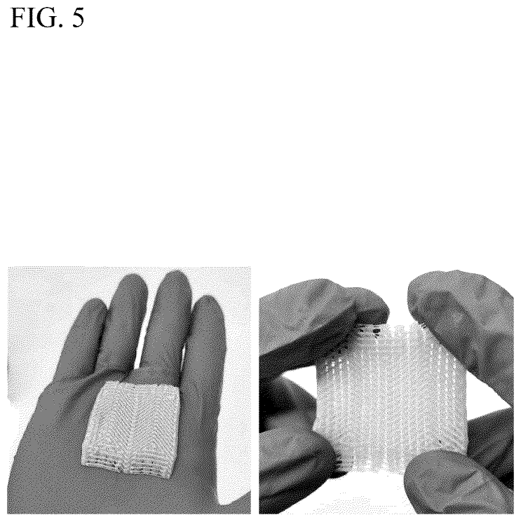

At this time, the printing pressure used was adjusted to 100 kPa, the speed was adjusted to 200 mm/min, and the temperature was adjusted to 25° C. The muscle cell tissue (Pattern A) and the connective tissue (Pattern B) illustrated in FIG. 1 were laminated by crossing each other once, and as illustrated in FIG. 3, the cell-based fish was printed in a structure in which adjacent patterns A were offset from each other. The laminated structure immediately after 3D printing are illustrated in FIG. 5.

### Step of Crosslinking Laminated Structure

The laminated structure illustrated in FIG. 5 was crosslinked using 150 mM edible calcium chloride (CaCl₂)) to maintain its shape, and was physically gelled in an incubator at 37° C. for 1 hour.

### Culturing Step

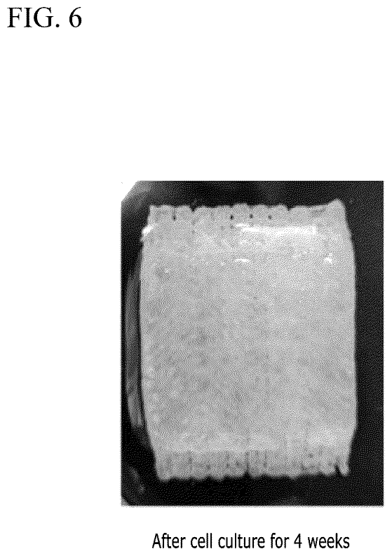

The gelled laminated structure was cultured in a bioreactor at 25° C. The mixed culture medium supplied to the bioreactor was automatically exchanged in the bioreactor, and cultured at 100 rpm, 25° C., pH 7.4 for 28 days. The cell-based fish with muscle tissue generated after cell culture for 4 weeks are illustrated in FIG. 6.

### Step of Processing and Cooking Cell-Based Fish

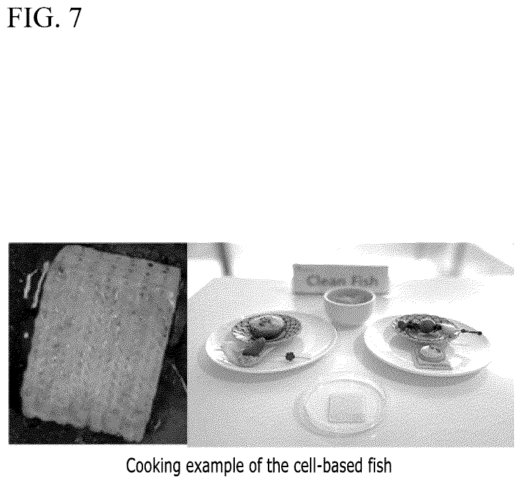

It could be confirmed that the cell-based fish illustrated in FIG. 6 could be rinsed in a portable water, and then cooked in various dishes such as frying or steaming as illustrated in FIG. 7, and even after cooking, the fish muscle shape-mimicking muscle remains intact, thereby maintaining the shape very similar to that of actual fish dishes.

### Measurement of Cell Alignment Degree: Measurement of Muscle Stem Cell Alignment Degree

To confirm the difference between a conventional method of using a scaffold, which is a general method of producing cell-based meat or cell-based fish, and a method for producing cell-based fish containing fish muscle shape-mimicking muscle using 3D bioprinting technology, the degree of muscle stem cell alignment was measured.

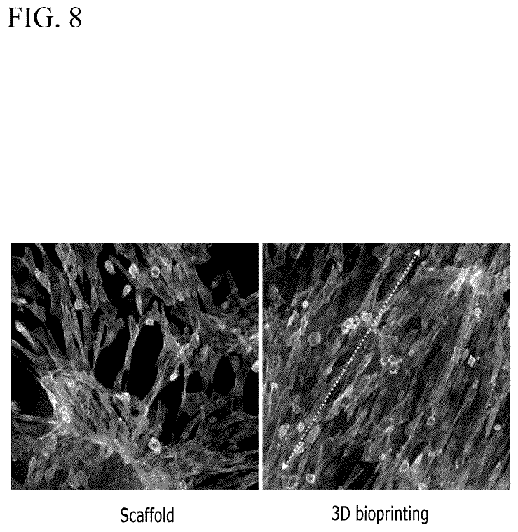

To measure the degree of muscle stem cell alignment within a scaffold fabricated by salt leaching and a cell-printed structure fabricated by bioprinting, the scaffold and cell-printed structure cultured for 28 days (4 weeks) were washed with PBS and carried and fixed on 4% paraformaldehyde (WAKO) for 20 minutes at room temperature. The fixed sample was washed three times with PBS, and then treated with a permeation solution (0.02% Triton X-100, 2% BSA in PBS) for 30 minutes at room temperature. Then, Phalloidin 488 (Abcam, 1:400) was diluted in PBS to stain the cell skeleton, DAPI (6-diamidino-2-phenylindole, Sigma, 1:1000) was diluted in PBS to stain the cell nucleus, and WGA (Wheat gem agglutinin (647), Invitrogen, 1 mg/ml) was diluted in PBS to stain a cell lectin, and stained for comparison for 1 hour at room temperature, and then fluorescence images were acquired using a fluorescence microscope. The results are illustrated in FIG. 8. As illustrated in FIG. 8, it can be seen that when a scaffold was used, muscle stem cells have a strong tendency to be randomly arranged in various directions. Therefore, when a scaffold was used, it can be confirmed that muscle tissue will ultimately be organized into a non-uniform shape. On the other hand, when 3D bioprinting technology is used, it can be confirmed that the muscle stem cells are aligned elongated in the direction in which they are ejected from a printer nozzle. Therefore, it can be confirmed that cell differentiation and organization occur in one direction, allowing muscle tissue to be formed close to actual fish muscle.

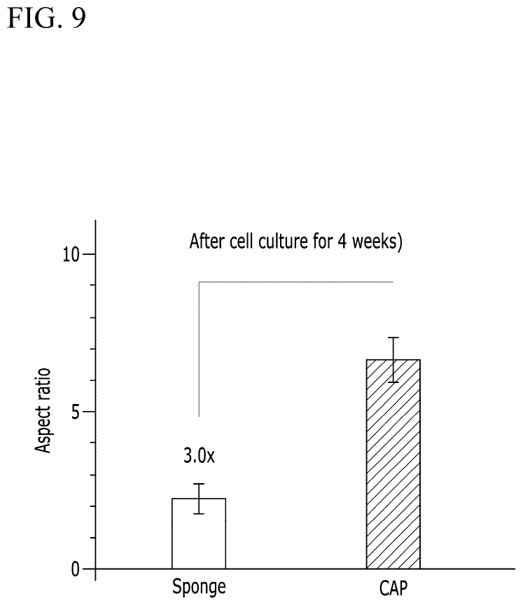

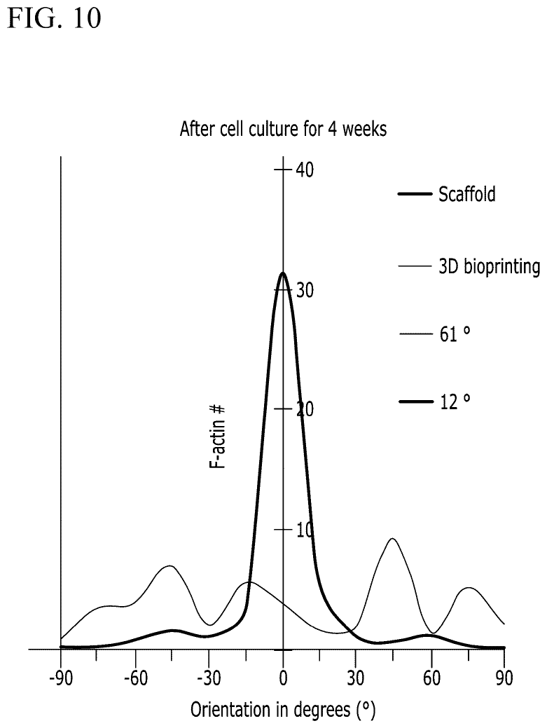

The results of calculating the cell aspect ratio and degree of alignment of cells from the acquired images using the Image-J (public domain) program are illustrated in FIGS. 9 and 10. Referring to FIG. 9, it can be confirmed that in the case of applying bioprinting (CAP), the cell aspect ratio is improved by more than 3 times as compared to the case of using a scaffold (sponge). Further, referring to FIG. 10, it can be confirmed that the alignment directivity is calculated as the full width at half maximum, and in the case of using a scaffold, the full width at half maximum (°) is 61°, whereas in the case of bioprinting, the alignment directivity is significantly improved to 12°.

### Measurement of Degree of Differentiation of Muscle Cells

To measure the degree of differentiation of muscle cells within a scaffold fabricated by salt leaching and a cell-printed structure fabricated by bioprinting, real-time PCR (real-time polymerase chain reaction) was performed to measure the mRNA expression levels of Myogenin, MyoD, and MyHC from scaffolds and cell-printed structures cultured for 28 days (4 weeks).

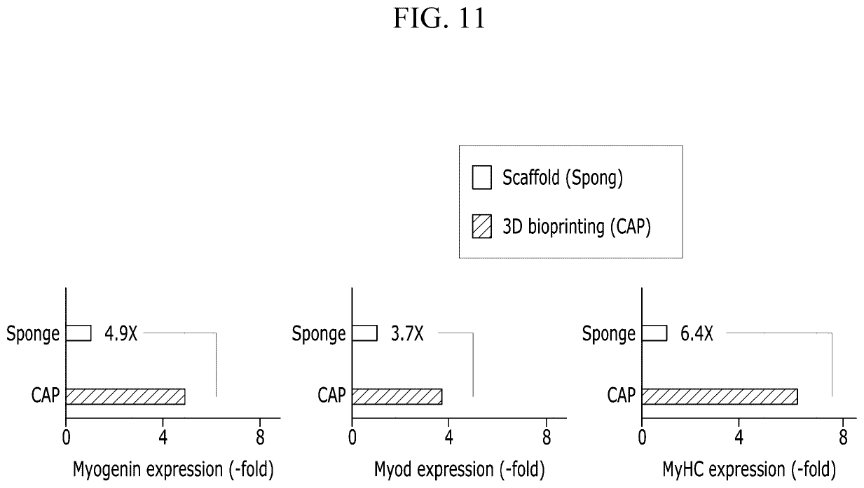

Each cell was collected from the sample, total RNA was isolated using TRI solution (Sigma-Aldrich), and the purity was measured using a spectrophotometer (INNO, LTECK). The cDNA synthesized using the reverse transcription system was measured using the StepOne Plus RT-PCR system (Applied Biosystems). The results are illustrated in FIG. 11.

From the results in FIG. 11, it can be confirmed that in the case of Myogenin, the expression level increased by about 4.9 times when applying bioprinting (CAP) as compared to when using a scaffold; in the case of Myod, the expression level increased by about 3.7 times when applying bioprinting (CAP) as compared to when using a scaffold; and in the case of MyHC, the expression level increased about 6.4 times when applying bioprinting (CAP) as compared to when using a scaffold.

From the above, it can be confirmed that when applying the method for producing cell-based fish by cell printing, the differentiation of muscle cells progresses much more easily, and it is possible to produce cell-based fish that mimics the muscle shape of actual fish and has the texture similar to that of actual fish.

### Measurement of Morphological Changes in Cells Caused by Edible Bioink Composition

To measure morphological changes in cells caused by the composition of edible bioink, myoblast cells extracted from Sebastes schlegeli were physically mixed with various bioinks at a concentration of 5×10⁷ cells/mL. The cells mixed with the bioink were ejected through a 20 G nozzle at a pneumatic pressure of 10 kPa, and cultured for 4 days. To observe the shape of cells on the 1^{st} and 4^{th} days of culture, the membranes of living cells were fluorescently stained with Calcein AM (Invitrogen, USA), and the nuclei of dead cells were fluorescently stained with Ethidium Homodimer-1 (Invitrogen, USA). The stained samples were photographed using an inverted fluorescence microscope (CKX53, OLYMPUS, Japan). The membranes of living cells were emitted at 517 nm by the stained Calcein AM and acquired as a green fluorescence image, and the nuclei of dead cells were emitted at 617 nm by EthD-1 and acquired as a red fluorescence image.

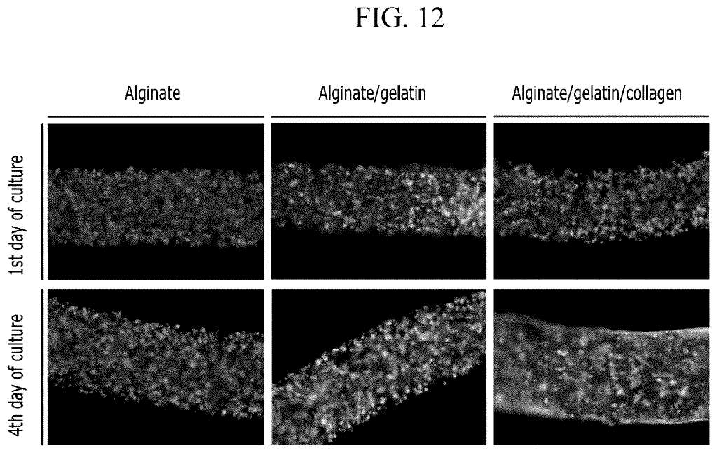

The fluorescence images measured on the 1^{st} day and the 4^{th} day of culture are illustrated in FIG. 12. From the results in FIG. 12, it can be confirmed that when gelatin was added or both gelatin and collagen were added, cell proliferation, adhesion, and mobility were improved, and cells were much better organized as compared to the case of using alginate alone.

### Measurement of Compressive Stress and Tensile Stress Applied to Muscle Tissue According to the Angle and the Line Width of the W Pattern

To measure the compressive stress, edible salmon, edible flounder, and W pattern laminated structures with an angle of 90 degrees and line widths of 0.4 mm, 0.6 mm, and 0.8 mm, respectively, were cut into a size of 15×15 mm to prepare a specimen. Then, the compressive stress was measured by applying a load of 50 kgf and a feed speed of 10 mm/min using a universal testing machine (QM100S, QMESYS, Gunpo, Korea). The compressive stress by strain rate was calculated through the manufacturer's software Qm_Tester.

To measure the tensile stress, the W pattern laminated structures with a line width of 0.6 mm and angles of 60 degrees, 90 degrees, and 120 degrees, respectively, were cut into a size of 10×0.2 mm to prepare a specimen. Then, the specimen was stretched using a universal testing machine (QM100S, QMESYS, Gunpo, Korea) at a force of 50 kgf and a feed rate of 10 mm/min to measure the tensile stress. The tensile stress by strain rate was calculated through the manufacturer's software Qm_Tester.

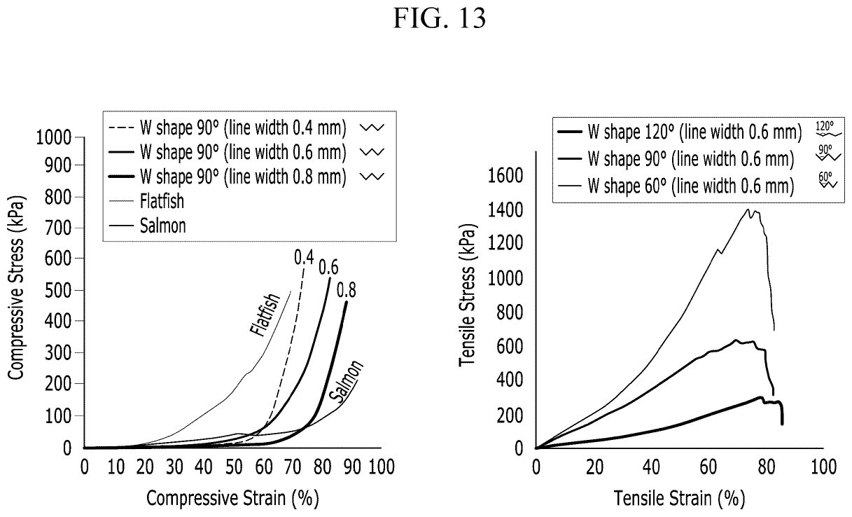

The results of measuring the compressive stress and tensile stress applied to muscle tissue formed by varying the angle and line width of the W shape are illustrated in FIG. 13. From the results in FIG. 13, it can be confirmed that as the line width increases, it exhibits a compressive stress to that of salmon, and as the angle of the W pattern decreases, the tensile stress increases. Therefore, it can be determined through extrapolation that the angle of the W pattern is preferably formed in the range of 20 to 70 degrees.

### Measurement of Cell Alignment (Organization) Based on Cell Concentration

To measure cell alignment based on cell concentration, myoblast cells extracted from Sebastes schlegeli were physically mixed with a bioink mixed with alginate, gelatin, and collagen at concentrations of 5×10⁷ cells/mL and 5×10⁸ cells/mL, respectively. The cells mixed with the bioink were ejected through a 27 G nozzle at a pneumatic pressure of 30 kPa, and cultured for 7 days. To observe the shape of cells on the 3rd and 7th days of culture, the membranes of living cells were fluorescently stained with Calcein AM (Invitrogen, USA), and the nuclei of dead cells were fluorescently stained with Ethidium Homodimer-1 (Invitrogen, USA). The stained samples were photographed using a confocal microscope (FV 1200, OLYMPUS, Japan). The membranes of living cells were emitted at 517 nm by the stained Calcein AM, and was acquired as a green fluorescence image, and the nuclei of dead cells were emitted at 617 nm by EthD-1, and was acquired as a red fluorescence image.

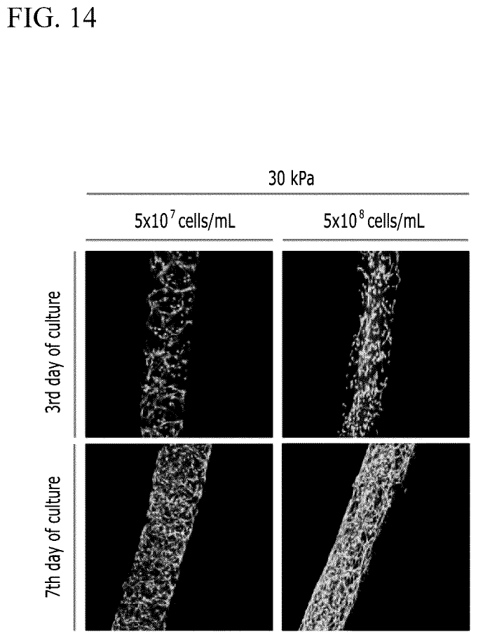

Fluorescence images measured on the 3rd day and the 7th day of culture are illustrated in FIG. 14. From the results in FIG. 14, it can be seen that cells are better organized into tissues in the case of 5×10⁸ cells/mL as compared to the case of 5×10⁷ cells/mL. From the results of the degree of organization in FIG. 14, it can be inferred that the lower limit of cell concentration is 1×10⁶ cells/mL and the upper limit is 1×10¹⁰ cells/mL.

### Measurement of Cell Alignment (Organization) Caused by Printing Pneumatic Pressure

To measure cell alignment caused by cell concentration, the myoblast cells extracted from Sebastes schlegeli were physically mixed with a bioink mixed with alginate, gelatin, and collagen at a concentration of 5×10⁶ cells/mL. The cells mixed with the bioink were ejected through a 27 G nozzle at a pneumatic pressures of 10 kPa and 60 kPa, respectively, and cultured for 7 days. To observe the shape of cells on the 3rd and 7th days of culture, the membranes of living cells were fluorescently stained with Calcein AM (Invitrogen, USA), and the nuclei of dead cells were fluorescently stained with Ethidium Homodimer-1 (Invitrogen, USA). The stained samples were photographed using a confocal microscope (FV 1200, OLYMPUS, Japan). The membranes of living cells were emitted at 517 nm the stained Calcein AM, and was acquired as a green fluorescence image, and the nuclei of dead cells were emitted at 617 nm by EthD-1, and was acquired as a red fluorescence image.

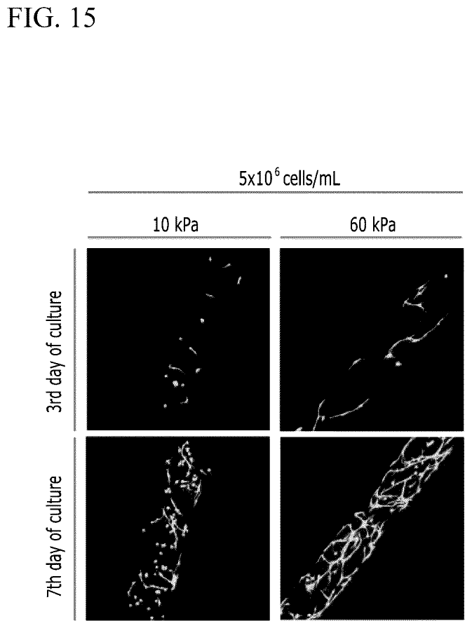

The fluorescence images measured on the 3rd day and 7th day of culture are illustrated in FIG. 15. From the results in FIG. 15, it can be confirmed that printing pneumatic pressure is an important factor affecting cell alignment.

Although the invention has been shown and described in detail with reference preferred embodiments thereof, the scope of the present disclosure is not limited thereto, and various modifications and improvements can be made by those skilled in the art using the basic concepts of the present disclosure which are defined in the appended claims, which also fall within the scope of the present disclosure.

### INDUSTRIAL APPLICABILITY

The present disclosure is applicable to the field of food production, especially the production of cell-based fish and the supply of cell-based fish.

1. A method for producing cell-based fish having fish muscle shape-mimicking muscle using 3D bioprinting technology, the method comprising the steps of:
mixing cells for cultured fish and an edible bioink to prepare a cell printing ink;
printing the cell printing ink in a fish muscle-mimic pattern to form a laminated structure;
adding a crosslinking agent to the laminated structure; and
culturing the laminated structure to which the crosslinking agent is added into a fish muscle tissue.

2. The method for producing cell-based fish according to claim 1,
wherein the step of culturing the laminated structure into a fish muscle tissue comprises,
aligning the cells in a direction in which they are ejected during printing within the bioink; and
forming a muscle tissue from the aligned cells.

3. The method for producing cell-based fish according to claim 1, wherein:
the cells for cultured fish are fish muscle stem cells or fish myoblast cells.

4. The method for producing cell-based fish according to claim 1, wherein:
the cells for cultured fish further comprise fish adipose cells or vascular endothelial cells.

5. The method for producing cell-based fish according to claim 1, wherein:
the cells for cultured fish are mixed with the edible bioink at a concentration of 1×10⁶ cells/mL to 1×10¹⁰ cells/mL.

6. The method for producing cell-based fish according to claim 1, wherein:
the fish muscle-mimic pattern is the W pattern.

7. The method for producing cell-based fish according to claim 6, wherein:
the laminated structure consists of a W pattern layer, which is a fish muscle-mimic pattern, and a line pattern layer that intersects with the W pattern.

8. The method for producing cell-based fish according to claim 7, wherein:
the W pattern layer and the line pattern layer are alternately laminated, or
repeatedly laminating the W pattern layer two or more times and then repeatedly laminating the line pattern layer two or more times are alternately performed.

9. The method for producing cell-based fish according to claim 7,
which performs printing so that the W pattern layer is located in the lowermost layer.

10. The method for producing cell-based fish according to claim 7, wherein:
the W pattern layers, which are adjacent vertically, are arranged offset from each other.

11. The method for producing cell-based fish according to claim 1, wherein:
the inner diameter of a nozzle during printing is 18G to 32G or 1.00 mm to 0.10 mm, or
the printing pneumatic pressure is 2 kPa to 500 kPa.

12. The method for producing cell-based fish according to claim 1, wherein:
a cell aspect ratio of the cell nuclei aligned within the cell-based fish is 2.0 to 5.0, or
a cell aspect ratio of the skeleton or cytoplasm of the cells aligned within the cell-based fish is 3.0 to 10.0.

13. The method for producing cell-based fish according to claim 1, wherein:
the full width at half maximum of the cells aligned within the cell-based fish is 1° to 20°.

14. The method for producing cell-based fish according to claim 1, wherein:
the edible bioink comprises alginate; and
one or more substances selected from the group consisting of collagen, gelatin and chitosan.

15. A cell-based fish with layer-by-layer laminated muscle tissue containing fish muscle-derived cells.

16. The cell-based fish according to claim 15, wherein:
the muscle tissue contains a W pattern muscle tissue that mimics fish muscle shape.

17. The cell-based fish according to claim 16, further comprising:
a connective tissue layer that intersects with the W pattern muscle tissue.

18. The cell-based fish according to claim 17, wherein:
the lowermost layer of the cell-based fish consists of the W pattern muscle tissue.

19. The cell-based fish according to claim 15, wherein:
a cell aspect ratio of the cell nuclei aligned within the cell-based fish is 2.0 to 5.0, or
a cell aspect ratio of the skeleton or cytoplasm of the cells aligned within the cell-based fish is 3.0 to 10.0.

20. The cell-based fish according to claim 15, wherein:
the full width at half maximum of the cells aligned within the cell-based fish is 1° to 20°.

21. The cell-based fish according to claim 15, wherein:
the cell-based fish comprises alginate; and
one or more substances selected from the group consisting of collagen, gelatin and chitosan.

22. The cell-based fish according to claim 15, further comprising:
fish adipose-derived cells or fish blood vessel-derived cells.
